(19) United States
(12) Patent Application Publication
(10) Pub. No.: US 2025/0268622 A1
(43) Pub. Date: Aug. 28, 2025

(54) CANNULA WITH IMPELLER PUMP, PERISTALTIC PUMP AND OTHER PUMPS

(71) Applicant: Edward J. Mikol, Pawleys Island, SC (US)

(72) Inventors: Edward J. Mikol, Pawleys Island, SC (US); Dirk V. Hoyns, Jackson, GA (US); Hung T. Than, Rockville, MD (US)

(21) Appl. No.: 19/065,730

(22) Filed: Feb. 27, 2025

Related U.S. Application Data
(60) Provisional application No. 63/558,205, filed on Feb. 27, 2024.

(51) Int. Cl. A61B 17/34; A61B 17/00

(52) U.S. Cl. CPC A61B 17/3421; A61B 2017/00398; A61B 2017/00557; A61B 2017/00862

(57) ABSTRACT

Disclosed herein is a surgical cannula having a cannula body defining a lumen and having a longitudinal axis, an outer membrane covering a portion of an outside surface of the cannula body, a pump fluidly connected to the lumen and to the outer membrane to transport a liquid from the lumen to inflate the outer membrane, and a dial rotatable to actuate the pump, wherein its axis of rotation is parallel to the longitudinal axis of the cannula body. The pump can be an impeller pump with a flexible impeller or a peristaltic pump. The surgical cannula can also be modular.

### FIELD OF THE INVENTION

The present invention is directed to a surgical cannula that utilizes a pump that after insertion or deployment into the patient's body or cavity at a surgical site transports liquid from the patient's body or cavity to inflate the outer membrane or balloon to anchor and/or seal the cannula to the surgical site and to deflate the outer membrane or balloon for removal after the surgical procedure. Preferably, the pump is an impeller pump, or alternatively a peristaltic pump or other pumps. The balloon may be a distal balloon and/or a balloon that covers most or some of the outer surface of the cannula body.

### BACKGROUND OF THE INVENTION

Cannulas have been used in minimally invasive surgical procedures, such as laparoscopic and arthroscopic surgeries. Typically, in these procedures a small incision made in the skin of a patient to access internal cavities, such as the abdomen or joints. A cannula is inserted into and is secured to the incision site. Surgical instruments are passed through the proximal openings of cannulas to enter a body cavity. During these procedures, the body cavity is inflated with an insufflated gas or liquid to create a surgical zone in the body cavity for surgical instruments. Insufflated gas or liquid can be pressurized up to 50 mm Hg or more. These cannulas generally have sealing members, such as an outer membrane or a distal balloon or umbrella, to seal the cannula to the incision site.

U.S. patent application publication No. 2009/0275898 to Wenchell discloses a cannula with an internal inflatable membrane in its lumen. Insufflated gas enters the proximal end of the cannula to inflate the internal membrane to seal the lumen with or without a medical instrument therein. However, the pressure within the inflated membrane with the insufflated gas would be the same as the pressure within the body cavity with the insufflated gas, i.e., both would have the same pressure of the insufflated gas. This is less than ideal because there is no positive pressure gradient from the inflated internal membrane to the body cavity for a positive seal.

U.S. Pat. No. 9,161,747 to Whittaker et al discloses a cannula with a plurality of protrusions located on the cannula's outer surface. These protrusions are extended outward against the incision site or cut tissue when a collar or a cam is rotated or a telescoping sleeve is pulled relative to the cannula. These anchoring protrusions are rigid and are pressed against the incision site, which may cause post-procedure discomfort for the patient.

U.S. patent Nos. and US published patent application Nos. U.S. Pat. Nos. 10,709,475; 10,687,848; US 2019/0274729; U.S. Pat. No. 11,202,654; US 2022/0110655 and US 2023/0037315 to Mikol et al disclose a number cannulas with passive priming mechanisms, with active pumping mechanisms and pumping with bellows, inter alia, to inflate the outer membrane or balloon to anchor and/or seal the cannula to the surgical site and to deflate the outer membrane of balloon for removal after the surgical procedure. These cannulas exhibit different mechanical mechanisms to inflate and deflate the balloons or outer membranes, and provide various options for cannula manufacturers. The Mikol patents and patent applications are incorporated herein by reference in their entireties.

There remains a need for additional mechanisms, such as pumps, to inflate the outer membrane or distal balloons on cannulas to achieve different goals or metrics, including but not limited to case of use, simpler designs, improved efficiencies, and meeting professional preferences of surgeons.

### SUMMARY OF THE INVENTION

Hence, the invention is directed to utilizing pumps, preferably mechanically operated pumps, in surgical cannulas to transport fluid, preferably the liquid inside body cavity or insufflated fluid, to inflate an outer membrane or distal balloon to anchor and/or seal the cannula to the cut tissue during surgical procedures. The pump actuator is preferably a rotatable dial, where the surgeons can rotate to transport fluid through the pump to inflate the outer membrane or distal balloon. Preferably, the axis of rotation of the dial is parallel to, preferably coincides with, the longitudinal axis of the cannula or the cannula body. The pump can be a flexible impeller pump or a peristaltic pump or other pumps.

These and other objects of the present invention are realized by a cannula comprising a cannula body defining a lumen and having a longitudinal axis, an outer membrane covering a portion of an outside surface of the cannula body, a pump fluidly connected to a port on the cannula body and to the outer membrane to transport a fluid through the port to inflate the outer membrane, and a dial rotatable to actuate the pump, wherein optionally its axis of rotation is parallel to or coincide with the longitudinal axis of the cannula body.

A rotation of the dial moves the fluid to inflate the outer membrane, and a counter-rotation of the dial reverses the flow of the fluid.

Preferably, the port opens to the lumen or to a distal end of the cannula body. Preferably, said port is fluidly connected, and more preferably connected, to an inlet of the pump.

The cannula further comprises a sealing cap with a diaphragm to cover the lumen. Preferably, the diaphragm comprises at least two disks each having a slit cut thereon, wherein the at least two disks are arranged so that the slits are at an angle to each other and wherein the slits are arranged to be aligned with the lumen. Also preferably, the diaphragm comprises at least two disks, wherein one disk has a slit cut thereon and the other disk has an opening that is sized to allow a medical instrument to pass through while maintaining a seal with an outer surface of the medical instrument.

In one embodiment, the outer membrane cover a significant portion of the cannula body, preferably more than a third, more preferably more than half of the cannula body. Preferably, the outer membrane has a dog bone or hour-glass shape. Also, preferably the outer membrane has an outer micro-textured surface, or the outer membrane has at least one macro texture such as a knob. Alternatively, the outer membrane is a distal balloon.

In one embodiment, the rotatable dial comprises an installed core and a removable dial, wherein the removable dial rotates a corresponding drive hub to operate the pump. Preferably, the removable dial is removed during a surgical procedure to minimize the cannula's dimensions.

In one embodiment, the pump is an impeller pump comprising a pump chamber having a cam surface including an outlet and an inlet, and an impeller comprising a plurality of flexible radial arms that contact the pump chamber, wherein a rotation of the dial rotates the impeller causing the fluid, such as insufflated fluid, to enter the pump chamber through the inlet and exit through the outlet, wherein the inlet is fluidly connected to said port.

Preferably, each flexible radial arm comprises an enlarged terminal in contact with the pump chamber. Preferably, the dial is keyed to the impeller to rotate the impeller as the dial is rotated.

In another embodiment, the pump is a peristaltic pump comprising a rotor member, sized and dimensioned to press a flexible tube containing a fluid, such as insufflated fluid, wherein the rotor comprises at least one arm/roller and wherein as the rotor rotates, its arm/roller pushes and presses against the flexible tube to move the fluid through the flexible tube and through the pump. Preferably, the dial is keyed to the rotor to rotate the impeller as the dial is rotated.

In one embodiment the pump is a peristaltic pump comprising a peristaltic chamber, a tubing assembly and a roller assembly, wherein the tubing assembly is positioned inside the peristaltic chamber and the roller assembly is located inside the tubing assembly. Preferably, the roller assembly comprises at least one roller rotatably mounted on a roller hub. Preferably, the tubing assembly comprises at least one tube fluidly connected to the lumen and the outer membrane. Preferably, when rotated the at least one roller presses the at least one tube to transport the fluid. Preferably, the peristaltic chamber comprises an inlet and an outlet, wherein the inlet is fluidly connected to the lumen or is located at a distal end of the cannula body and the outlet is fluidly connected to the outer membrane, wherein the inlet is fluidly connected to said port. Preferably, the dial is keyed to the roller assembly to rotate the roller assembly as the dial is rotated.

In one embodiment, the pump is modular, wherein the pump and the dial form a first modular part and a second modular part comprises the cannula body, the outer membrane and a pump receiving receptacle.

As used herein, cut tissues are the tissues cut by the surgeon during the operation to insert the cannula/trocar, and do not include the patient's skin surrounding the cut tissue and do not include the tissues untouched by the surgeon's scalpel surrounding the cut tissues.

Any and all embodiments of the inventive cannula may comprise a lumen seal positioned proximate to a distal end of the cannula body.

### BRIEF DESCRIPTION OF THE DRAWINGS

In the accompanying drawings, which form a part of the specification and are to be read in conjunction therewith and in which like reference numerals are used to indicate like parts in the various views:

FIG. 1 is a bottom perspective view of a preferred embodiment of an inventive cannula with an impeller pump;

FIG. 2 is an exploded view of the cannula of FIG. 1;

FIGS. 3(a) and 3(b) are a top and bottom exploded view, respectively, of a top or sealing portion of the cannula of FIG. 1;

FIG. 4(a) is an exploded view of the impeller pump portion of the cannula of FIG. 1; FIG. 4(b) is an exploded view of the impeller of the cannula of FIG. 1;

FIG. 5 is a top view of the impeller pump of the cannula of FIG. 1 with the impeller cap omitted for clarity;

FIG. 6 is a top perspective view of the cannula body;

FIGS. 7(a) and 7(b) are a side view and bottom view, respectively, of the cannula of FIG. 1, FIG. 7(c) is a cross-sectional view along line A-A, and FIG. 7(d) is an enlarged view of a portion of FIG. 7(c);

FIGS. 8(a) and 8(b) are a side view and bottom view at a different angle, respectively, of the cannula of FIG. 1, FIG. 8(c) is a cross-sectional view along line B-B, and FIG. 8(d) is an enlarged view of a portion of FIG. 8(c);

FIG. 9 is a partial exploded view of the cannula of FIG. 1, including an enlarged portion of the impeller;

FIG. 10 is a bottom perspective view of a preferred embodiment of an inventive cannula with a peristaltic pump;

FIG. 11 is an exploded view of the cannula of FIG. 10;

FIGS. 12(a) and 12(b) are a top and bottom exploded view, respectively, of a top or sealing portion of the cannula of FIG. 10;

FIGS. 13(a) and 13(b) are a top and bottom exploded view, respectively, of the pump drive dial and peristaltic roller assembly of the cannula of FIG. 10;

FIG. 14(a) is an exploded view of the peristaltic pump chamber including the peristaltic roller assembly of FIGS. 13(a)-(b) and the peristaltic tubing manifold; FIGS. 14(b) and (c) are a top and bottom view, respectively, of the peristaltic pump chamber of FIG. 14(a) when assembled;

FIGS. 15(a)-(d) are various views of the peristaltic tubing manifold with partial enlarged views of the tubes and a tubing manifold in FIG. 15(b);

FIGS. 16(a) and (b) are a bottom and top perspective view, respectively, of the cannula body; FIG. 16(c) is a bottom view thereof; FIG. 16(e) is a cross-sectional view of the cannula body long line B-B of FIG. 16(d); and

FIG. 17(a) is a front view of the cannula of FIG. 10; FIG. 17(b) is a cross-sectional view along line C-C in FIG. 17(a); FIG. 17(c) is a bottom view thereof;

FIG. 18(a) is a top perspective view of an alternative drive dial for the cannulas described herein; FIG. 18(b) is a partial top exploded view of the cannula of FIG. 18(a) showing a removable drive dial; FIG. 18(c) is a partial bottom exploded view thereof with a partial enlarged view of the removal drive dial;

FIG. 19(a) is a side view of another cannula of the present invention; FIGS. 19(b)-(c) are a top and bottom perspective view, respectively, of the cannula of FIG. 19(a) with the modular pump separated from the rest of the cannula assembly; and

FIGS. 20(a)-(b) are a top and bottom exploded view, respectively, of the cannula of FIG. 19(a) showing the component of the cannula.

### DETAILED DESCRIPTION OF THE PREFERRED EMBODIMENTS

The present invention is directed to utilizing pumps, preferably mechanically operated pumps, in surgical cannulas to transport fluid, preferably the liquid inside body cavity or insufflated fluid, to inflate an outer membrane or distal balloon to anchor and/or seal the cannula to the cut tissue during surgical procedures. The pump actuator is preferably a rotatable dial, where the surgeons can rotate to transport fluid through the pump to inflate the outer membrane or distal balloon. Preferably, the axis of rotation of the dial is parallel to the longitudinal axis of the cannula or the cannula body. More preferably, the axis of rotation of the dial coincides with the longitudinal axis of the cannula or the cannula body.

A preferred pump is an impeller pump. Another preferred pump is a peristaltic pump.

Impeller pumps have been utilized in industrial and heavy machinery settings, and are typically operated by electric motors at high rotational speeds to transport liquids from one place to another. Impeller pumps are used in the oil and gas industry and as bilge pumps in marine settings, among other usages. Impeller pumps operate by rotating an impeller comprising a central hub with blades or arms emanating therefrom. The rotation creates a zone of low pressure and a zone of high pressure to move liquid through the pump. There are generally two types of impeller pumps, one with a rigid impeller and one with a flexible impeller and an internal cam section to bend the flexible impeller.

In pumps with rigid impeller, the inlet is located proximate the center of rotation and the outlet is located at the periphery of the pump. Liquid flows into the inlet and is pushed outward by the rotating rigid impeller thereby gaining speed toward the outlet.

In pumps with flexible impeller, the arms on the impeller contact the internal surface of the pump housing. When the arms on impeller are rotated towards the internal cam section, the arms are bent by the cam reducing the volume the area between adjacent arms to increase pressure. As the arms pass the cam section this volume expands to lower pressure. The inlet is located at the area with reduced pressure and the outlet is located at the increased pressure. Both inlet and outlet are on the periphery of the pump.

In a preferred embodiment, flexible impeller pumps are adapted for surgical cannulas. The operating environment of surgeries is different than those in industrial settings in that the pumps are necessarily smaller and operating in significantly lower pressures and that the pumps are preferably operated by hand instead of an electrical motor. Alternatively, the flexible impeller pumps, as well as the peristaltic pump, may be operated by an electrical motor sized and dimensioned to rotate the pump's drive dial.

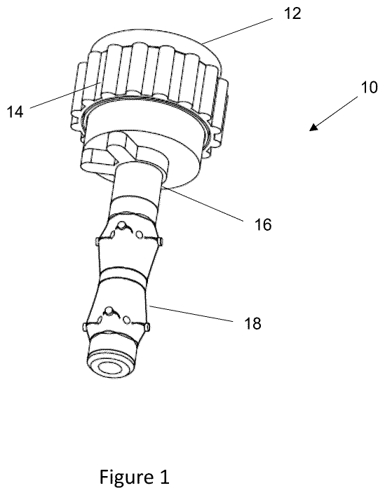

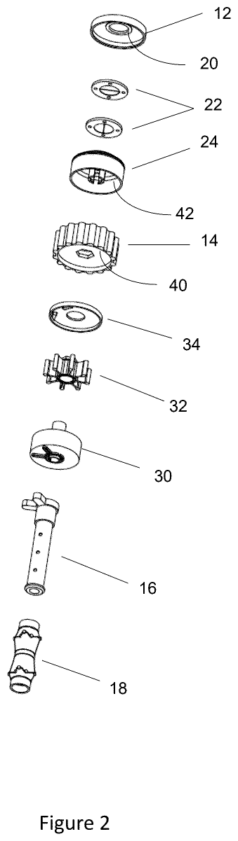

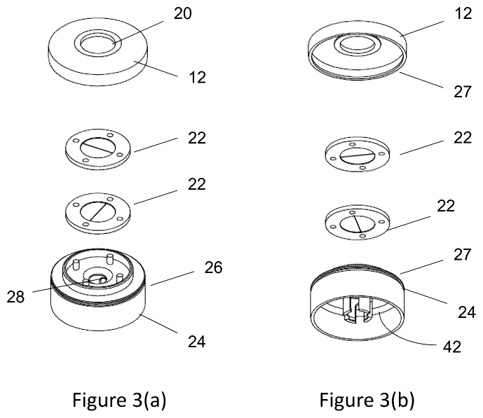

An exemplary inventive cannula 10 is illustrated in FIG. 1. As shown, cannula 10 has a proximal seal cap 12, a pump actuator, i.e., impeller drive dial 14 with a cannula body 16 defining a lumen and an inflatable outer membrane 18, which is inflated as shown in FIG. 1. The components of cannula 10 are shown in the exploded view of FIG. 2 illustrating all components arranged to assemble together to form the cannula. FIGS. 3-9 illustrate in detail subsets of these components to convey their functions and structures.

Referring to FIGS. 2, 3(a) and 3(b), cap 12 defines an opening 20, sized and dimensioned to allow surgical equipment to pass through the cap and the lumen 60 defined by cannula body 16 into the patient's body. A diaphragm is provided to close the lumen while medical instruments are not used, keeping the cavity pressurized. The diaphragm preferably comprises two instrument seals 22. Each seal 22 comprises a slit and the seals are preferably arranged so that the slits are at an angle to each other, preferably at a right angle to each other, as shown. The seals 22 has four peripheral holes that align to four corresponding locating pins on a seal retainer 24. When assembled, the seals are aligned as shown and fit onto the locating pins and cap 12 fitted onto retainer 24. The cap can be press-fitted or adhesively attached to retainer 24, or preferably snap-fitted using snap element 26 on seal retainer 24 and snap element 27 on cap 12, as shown. Retainer 24 also defines an opening 28 aligned with opening 20. Other diaphragm designs are contemplated. For example, the diaphragm may comprise a single seal 22, triple seal, or a seal with perpendicular or cross slits.

Referring to FIGS. 2, 4(a) and 4(b) and 5, below retainer 24 is the flexible impeller pump, which comprises pump chamber 30, flexible impeller 32 and pump chamber cap 34. Flexible impeller 32 is rotatably received on axle 36 and cap 34 closes the pump chamber. Drive hub 38 is configured to rotate impeller 32 when drive hub 38 is rotated. In this embodiment, drive hub 38 is driven by impeller drive dial 14. Drive hub 38 protrudes above chamber cap 34 when assembled.

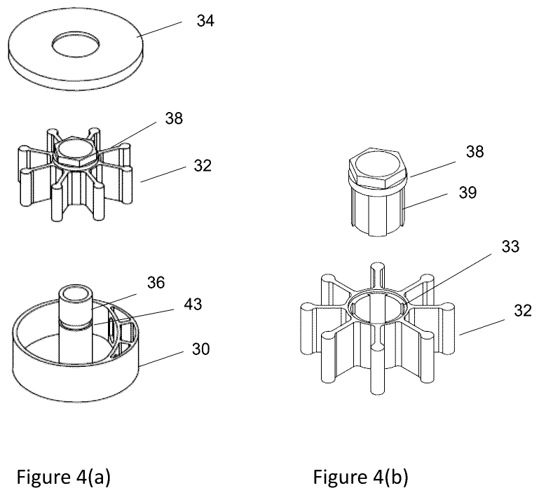

Drive hub 38 and impeller drive dial 14 may be keyed in any known manner. As shown, drive hub 38 is a polygonal shaped nut, which is received in driving hole 40, which has a corresponding polygonal shape, located on drive dial 14. Referring to FIGS. 2 and 3(b), seal retainer 24 has a snap connector 42 that is sized and dimensioned to be inserted through impeller drive dial 14 and snap-fitted to snap groove 43 located on axle 36 to close pump chamber 30. As best shown in FIG. 4(b), drive hub 38 comprises a keyed shaft 39, which is received in keyed opening 33 of impeller 32 to fix drive hub 39 to impeller 32, so that they rotate together.

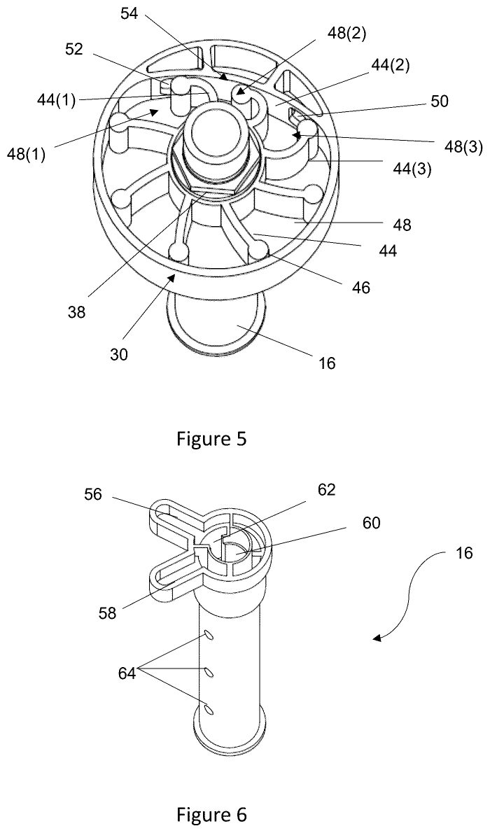

Pump chamber 30 is shown in detail in FIG. 5, with the chamber cap 34 and impeller drive dial 14 omitted for clarity. Flexible impeller 32 has a number of arms 44 with enlarged terminals or ends 46 which maintain contact with the inside surface of pump chamber 30 while rotating. Segments 48 are defined as the volumes between adjacent arms 44, the bottom surface of chamber 30 and chamber cap 34. Segments 48 are substantially sealed and preferably sealed while rotating to maintain the variation of pressures within each segment 48 caused by the changes in the volume in each segment.

Pump chamber 30 also has inlet port 50 and outlet port 52 defined on cam surface 54. Inlet port 50 is fluidly connected to the liquid in the body cavity, and outlet port 52 is fluidly connected to outer membrane 18. As impeller 32 is rotated, clockwise as shown in FIG. 5, as terminal 46 of arm 44(1) contacts and moves across cam surface 54, the volume of segment 48(1) is reduced thereby increasing the pressure within segment 48(1) to push liquid out through outlet port 52 to inflate outer membrane 18. Concurrently, the volume of segment 48(2) is further reduced by the bending of arm 44(1), and as arm 44(3) loses contact with cam surface 54 it straightens and the volume of segment 48(3) increases thereby reducing its pressure. This reduction in pressure draws liquid from the body cavity through inlet port 50. This inflow and outflow cycle repeats as impeller 32 continues to rotate to operate the impeller pump.

As discussed above, in surgical applications the liquid in the body cavity is pressurized up to 50 mm Hg or about 1 psi, possible more. At that pressure, the body cavity liquid would flow into segment 48(3) as its volume increases with or without a decrease in the pressure in segment 48(3).

Also, as shown in FIG. 5, the volume of segment 48(2) is reduced due to cam surface 54's protrusion into pump chamber 30 thereby increasing its pressure. Arm 44(1) is bent in a backward orientation due to the clockwise rotation of impeller 32 and since liquid is essentially incompressible, a portion of the liquid in segment 48(2) would flow into segment 48(1) further pressing arm 44(1) backward. Liquid would not flow into segment 48(3) because the increased pressure applies to the back side/surface of arm 44(2), and increased pressure within segment 48(2) would push terminal 46 of arm 44(2) into a sealing position with cam surface 54. On the other hand, increased pressure in segment 48(2) would apply to the front side/surface of arm 44(1) and tend to push terminal 46 of arm 44(1) away from cam surface 54, allowing liquid in segment 48(2) to enter segment 48(1) and likely outward through outlet port 52.

Referring to FIGS. 2 and 6, below or distally from pump chamber 30 is cannula body 16. Body 16 comprises an inlet chamber 56 in fluid communication with inlet port 50 and an outlet chamber 58 in fluid communication with outlet port 52. Body 16 defines lumen 60 which is fluidly connected to inlet chamber 56 via inlet duct 62. Surgical instruments pass through lumen 60 to the body cavity and insufflated liquid from the body cavity fills lumen 60. Outlet port 52 and outlet chamber 58 are fluidly connected to outer membrane 18 through inflation holes 64.

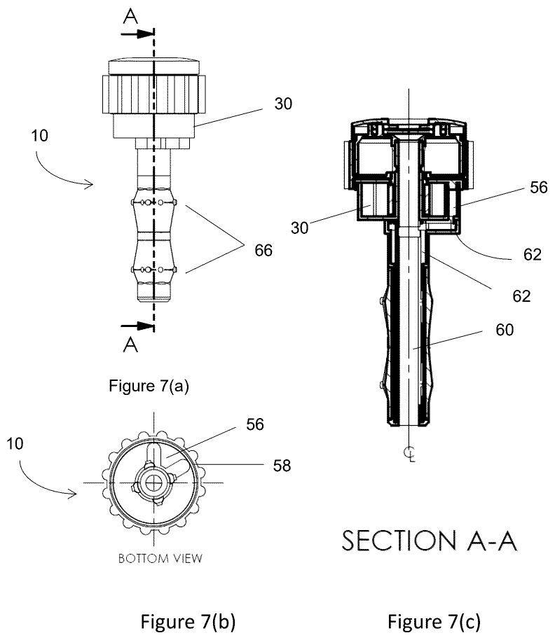

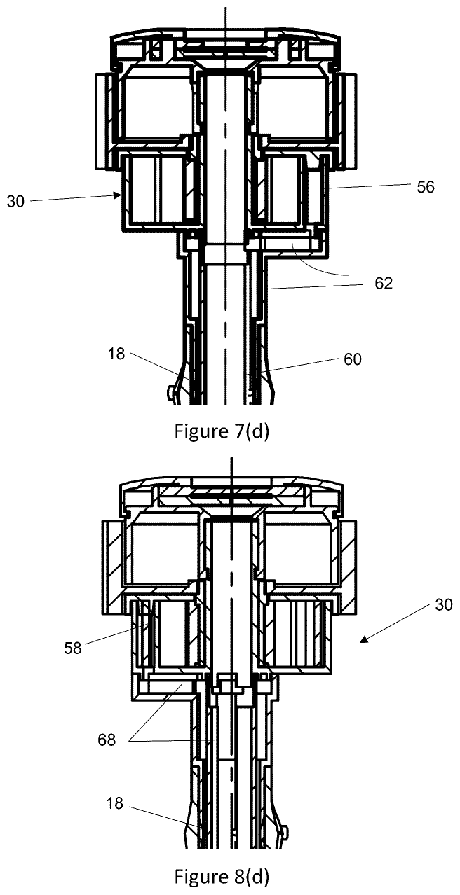

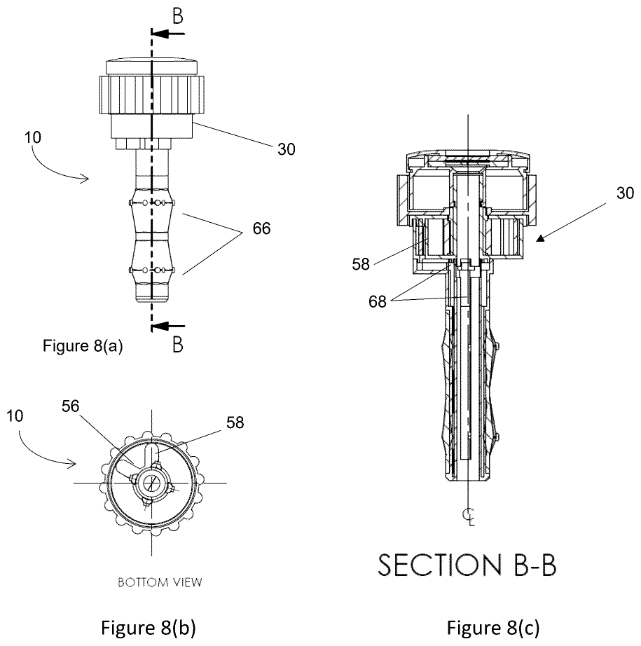

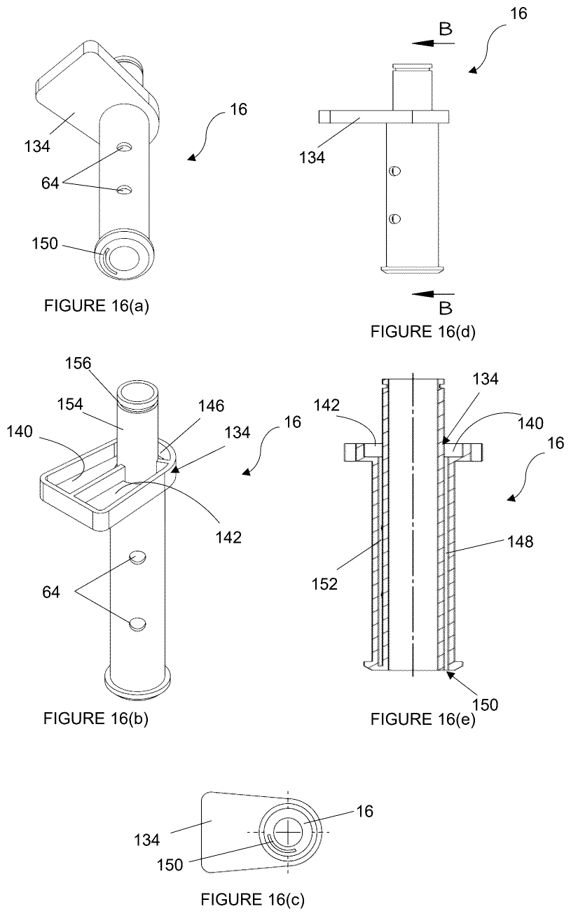

FIGS. 7(a)-7(c) show the flow path from lumen 60 to impeller pump 30 through inlet duct 62 and inlet chamber 56. Inlet duct 62 ends in a port that opens to lumen 60. It is noted that inlet duct 62 may extend distally to the distal terminus of cannula body 16, similar to inlet channel 148 and distal port/terminal 150 shown below in FIGS. 16(a)-(e) and 17(c) in connection with cannula 100. FIGS. 8(a)-8(c) show the flow path from outlet chamber 58 of pump chamber 30 through outlet duct 68 to inflation holes 64 to inflate outer membrane 18. As shown, inlet duct 62 and outlet duct 68 have a longitudinal component and a transverse component. FIGS. 7(d) and 8(d) are enlarged views of a portion of FIGS. 7(c) and 8(c), respectively, showing the flow paths.

As best shown in FIGS. 7(a) and 7(b), the lines marked as lines A-A and B-B are the longitudinal axis of cannula 10 or cannula body 16. The axis of rotation of drive dial 14 and impeller 32 is parallel to this longitudinal axis and preferably coincides with this longitudinal axis. The center line (℄) in FIGS. 7(c) and 8(c) is also the longitudinal axis.

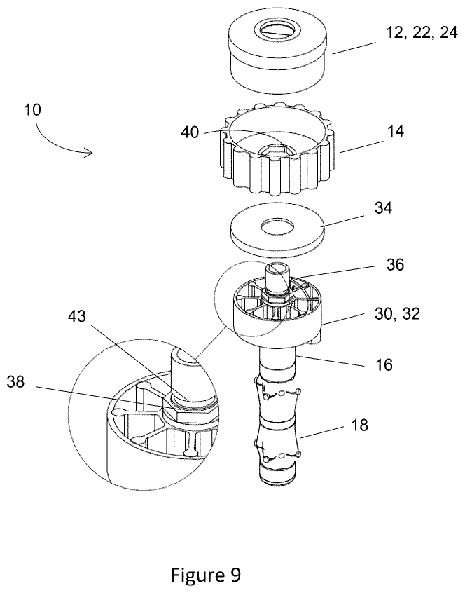

FIG. 9 shows a partially assembled cannula 10 to illustrate the assembly of the various components. Cap 12, seals 22 and cap retainer 24 are connected via snaps 26 and 27, and are collectively received by drive dial 14. Snap connector 42 located under cap retainer 24, as best shown in FIG. 3(b), is connected to snap groove 43 located on axle 36 to secure drive dial 14 and pump chamber cap 34 to pump chamber 30, thereby completing the assembly of cannula 10. When assembled, driving hole 40 on drive dial 14 fits onto drive hub 38, and rotating drive hub 14 rotates drive hub 38 and impeller 32.

In the configuration shown the drawings and as illustrated in FIG. 5, a clockwise rotation of impeller 32 by drive dial 14 inflates outer membrane 18. A counterclockwise rotation, i.e., a counter-rotation, of impeller 32 deflates outer membrane 18 by reversing the direction of flow through the pump.

The present inventor has determined that outer membrane 18 preferably has a dog bone or hourglass or dumbbell shape to improve the cannula's attachment to the cut tissue that cannula 10 is inserted into. Outer membrane 18 preferably also contains a plurality of knobs 66 that are preferably located on both sides of the waist section. When cannula 10 is inserted and outer membrane is inflated, knobs 66 extend into the cut tissue to secure cannula 10 to the cut tissue. As discussed in the Mikol patents and patent applications which have been incorporated by reference, the pressure inside inflated outer membrane 18 can be increased above the pressure of the insufflated liquid inside the body cavity. This creates a higher pressure zone between the body cavity and outside the body, which tends to secure the cannula to the cut tissue better. The inflated outer membrane also seals the space between the cannula and the cut tissue thereby reducing incidents of blood or other bodily fluids leaking by going around the cannula to the outside. Optionally, the outer surface of outer membrane 18 may also be textured, e.g., including micro-structures, to improve the attachment of the inflated cannula to the cut tissue. As shown in the figures, outer membrane 18 is not inflated. Knobs 66 can be omitted.

The dog bone shape advantageously minimizes a tendency that the outer membrane may herniate into the body cavity or out of the skin along the paths of least resistance. That dog bone shape helps to keep the balloon expanding axially between the ends of the balloon and prevents it from expanding along the axis of the cannula. The present invention covers outer membranes of any shape, including but not limited to the dog bone shape and the shapes that are known heretofore.

In another embodiment, a peristaltic pump is utilized instead of the impeller pump.

Peristaltic pumps for used in research settings are described in literature and in patent documents. An example is shown in U.S. published patent application no. US 2006/0198763, which is incorporated herein by reference in its entirety. Generally, a peristaltic pump comprises a rotor member, sized and dimensioned to press a flexible tube, which would contain a liquid, such as insufflated fluid. As the rotor member rotates, its arm(s) pushes and presses against the flexible tube to move liquid through the tube and thereby through the pump.

Pump chamber 30 may be modified to house a peristaltic pump. For example, cam surface 54 is omitted, and the flexible tube containing insufflated fluid is coiled along the inside wall of pump chamber 30. Impeller 32 is replaced by a rigid rotor with at least one radial arm that is sized and dimensioned to press on the flexible tube to move liquid to outer membrane 18. Drive dial 14 can be adapted to rotate the rotor in the peristaltic pump.

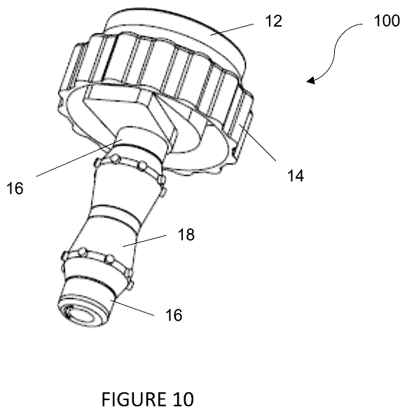

An exemplary, non-limiting cannula 100 utilizing a peristaltic pump is shown in FIGS. 10-17 and subparts. For simplicity, elements from cannula 100 that are the same or similar to those in cannula 10 shown in FIGS. 1-9 and subparts have the same reference numbers. Similar to cannula 10, cannula 100 also has proximal seal cap 12, pump drive dial 14, cannula body 16 and inflatable outer membrane 18, as illustrated in FIG. 10.

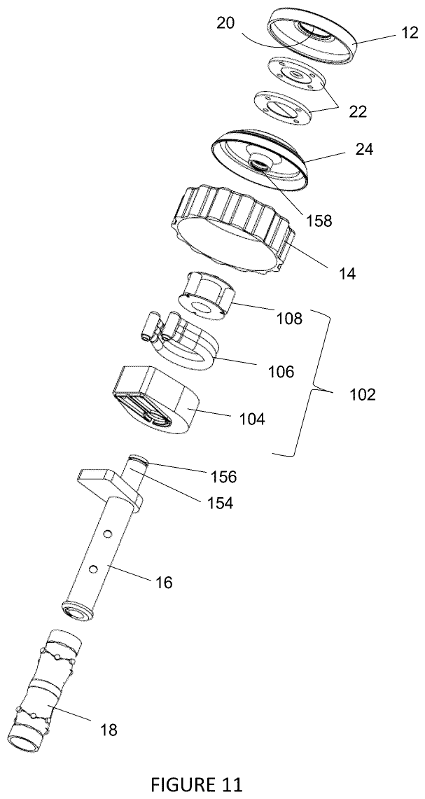

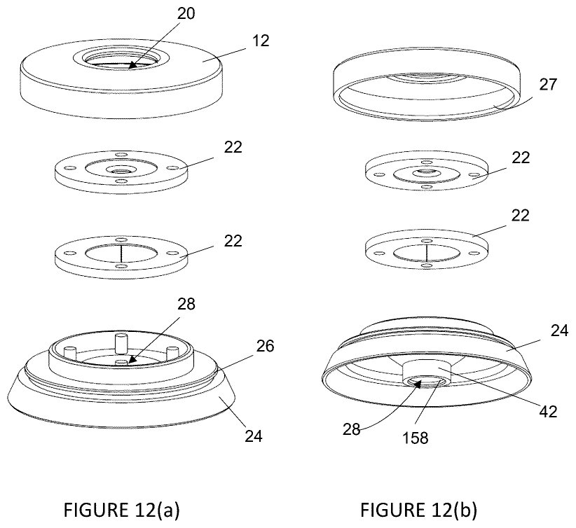

Proximal seal cap 12 for cannula 100 shown in FIGS. 11 and 12(a)-(b) is similar in structure and function as seal cap 12 for cannula 10 shown in FIGS. 3(a)-(b), except as noted. Both seal caps 12 have top opening 20 and bottom opening 28 on snap connector 42 on retainer 24 that align to each other to allow the passing of medical instruments through the cannula. Both seal caps 12 have at least one, preferably two, instrument seals 22 with at least one slit defined thereon. As shown, at least one diaphragm 22 has an opening that is sized to allow a medical instrument to pass through while maintaining a seal with an outer surface of the medical instrument. The other diaphragm has a slit. The proximal seal cap shown in FIGS. 12(a)-(b) can be used with cannula 10; and the proximal seal cap shown in FIGS. 3(a)-(b) can be used with cannula 100.

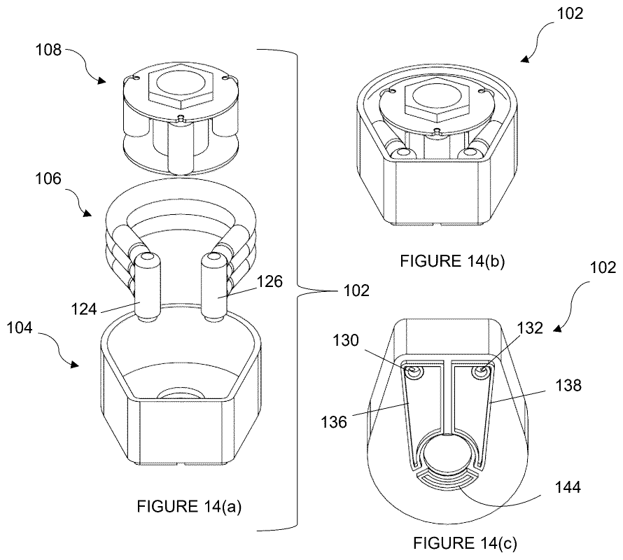

Referring to FIGS. 11, 13 and 14 and their subparts, below seal retainer 24 is peristaltic pump 102 which comprises peristaltic chamber 104, tubing assembly 106 and roller assembly 108, as best illustrated in FIGS. 11 and 14(a).

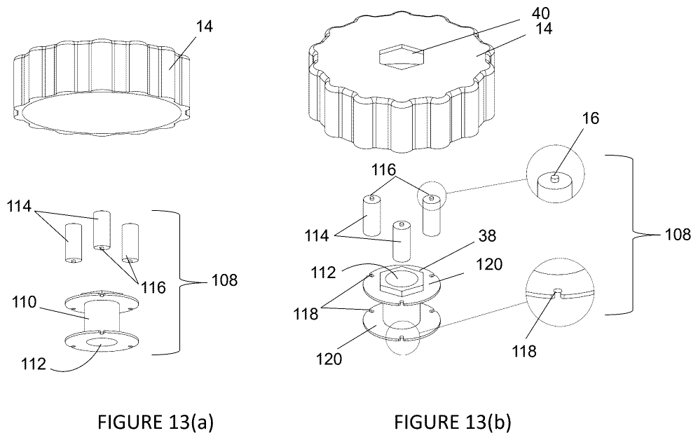

Roller assembly 108, as best shown in FIGS. 13(a)-(b) with drive dial 14, comprises roller hub 110 defining a cannula channel 112 for medical instruments to pass through the cannula. A plurality of pump rollers 114, shown with three rollers but any number can be used, are rotatably mounted to roller hub 110. Each roller 114 has a top and bottom axles 116, which are connected to roller axle snap connectors 118 provided at the top and bottom flanges 120 of roller hub 110. Roller assembly 108 also comprises drive hub 38, which may be a hexagonal-shaped nut as shown. Drive hub 38, when cannula 100 is assembled, is received in driving hole 40 on drive dial 14, so that rotation of drive dial 14 rotates roller assembly 108 and rollers 114.

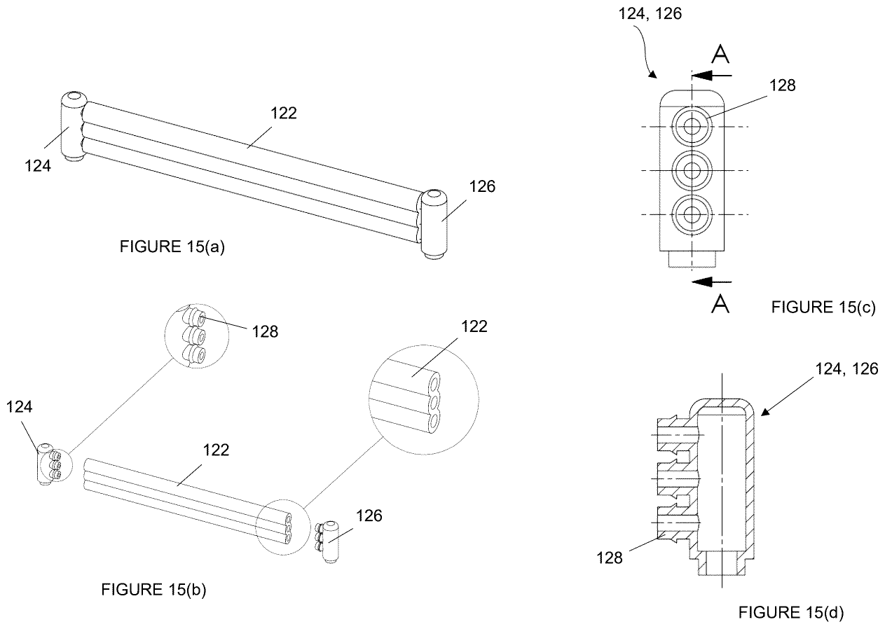

Tubing assembly 106 is best illustrated in FIGS. 15(a)-(d). The tubing assembly has a plurality of tubes 122. While three tubes 122 are shown, any number of tubes can be used. Tubes 122 are connected at each end to tubing manifold 124, 126. One tubing manifold is an inflow manifold and the other is an outflow manifold, and are further described below. Both manifolds have similar or same structures, as best illustrated in FIGS. 15(c)-(d). Each manifold has a plurality of fitting barbs 128, which are sized and dimensioned to fluidly connect to tubes 122, as shown, and preferably provide fluid seals between the tubing manifolds 124, 126 and tubes 122.

Referring to FIGS. 14(a)-(c), tubing assembly 106 when assembled has a horse-shoe shape; however, tubing manifold can have any circular shape or any shape with at least one circular portion. Tubing assembly 106 is placed within peristaltic chamber 104, and roller assembly 108 is placed inside tubing assembly 106. As best shown in FIG. 14(b), when assembled tubes 122 are flushed against the inside wall of peristaltic chamber 104 and rollers 114 are pressed into tubes 122. When roller assembly 108 is rotated by drive dial 14, rollers 114 compress tubes 122 and fluid within tubes 122 is pushed in the direction of the movement of rollers 114. As discussed further below, the movement of rollers 114 in one direction moves fluid to inflate outer membrane 18 or distal balloon and movement in the opposite direction deflate the outer membrane or balloon. As best shown in FIG. 14(c), an inlet 130 on the bottom of peristaltic chamber 104 is fluidly connected to inlet manifold 124. Outlet 132 also on the bottom of peristaltic chamber 104 is connected to outlet manifold 126. During cannula insertion, fluid from the body cavity such as insufflated fluid enters inlet 130 and is pumped into tubing assembly 106 at inlet manifold 124 and out through outlet manifold 126 to expand outer membrane 18 or a distal balloon. During cannula withdrawal, fluid moves in the opposite direction to deflate outer membrane or distal balloon 18.

Cannula body 16 is best shown in FIGS. 11 and 16(a)-(e). Cannula body 16 has a receiving tray 134, which is sized and dimensioned to receive peristaltic chamber 104. Referring to FIGS. 14(c) and 16(b), peristaltic chamber 104 on its bottom surface has raised edge or lip 136 around inlet 130 and raised edge or lip 138 around outlet 132. Edge 136 is received in a scaling manner in inlet reservoir 140, and edge 138 is received in a sealing manner in outlet reservoir 142. Edges 136 and 138 seal inlet and outlet reservoirs 140, 142, respectively, to isolate the inflow from the outflow in the peristaltic pump. Optionally, raised edge 144 on the bottom of peristaltic chamber 104 is provided and is received in chamber 146 on receiving tray 134 to help align peristaltic chamber 104 to receiving tray 134.

Within the wall of cannula body 16, as best shown in FIG. 16(e), in fluid communication with inlet reservoir 140 and inlet manifold 124 is body inlet channel 148 with distal terminal 150. Body inlet channel 148 is in fluid communication with the body cavity and/or the insufflated fluid, which is preferably used to inflate outer membrane 18 or a distal balloon. In fluid communication with outlet reservoir 142 and outlet manifold 126 is body outlet channel 152, which is fluidly connected to inflation hole(s) 64. It is noted that body inlet channel 148 may ends within lumen 60 and terminal/port 150 can be a port that opens into the lumen, similar to inlet duct 62 discussed in connection with cannula 10 and shown in FIG. 7 and subparts.

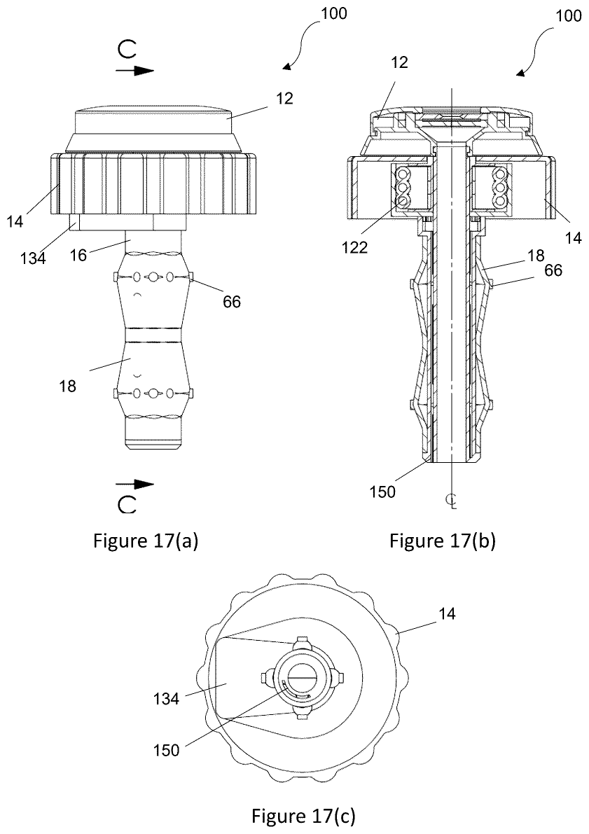

The cannula parts shown in FIG. 11 are connected to assemble cannula 100, which is shown in FIGS. 11 and 17(a)-(c). To assemble cannula 100, outer membrane 18 is pulled or positioned on the outside of cannula body 16 covering inflation hole(s) 64. Assembled peristaltic pump 102 is inserted onto upper shaft 154 of cannula body 16. Snap fitting 156 on upper shaft 154 is snap fitted to matching snap fitting 158 on connector 42 of seal retainer 24. Seal retainer 12 is connected by snap elements 26, 27 discussed above. As drive dial 14 is turned by a surgeon in the direction from inlet 130 toward outlet 132, rollers 114 presses tubes 122 and pushes fluid contained in tubes 122 toward outlet 132 and inflation holes 64 to inflate outer membrane 18. At the same time, the movements of rollers 114 creates a partial vacuum or reduced pressure in inlet 130 to draw additional fluid from the body cavity into tubes 122 to continue the pumping action until outer membrane 18 is fully inflated. A counter-rotation in the reverse direction would deflate outer membrane 18 and cannula 110 can be withdrawn.

Peristaltic pump 102 can be readily primed when distal terminal 150 is immersed in the fluid in the body cavity or in the insufflated fluid and drive dial 14 is rotated until the fluid reaches the outer membrane.

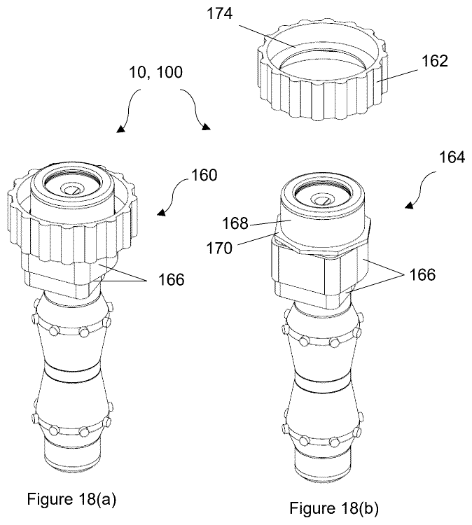

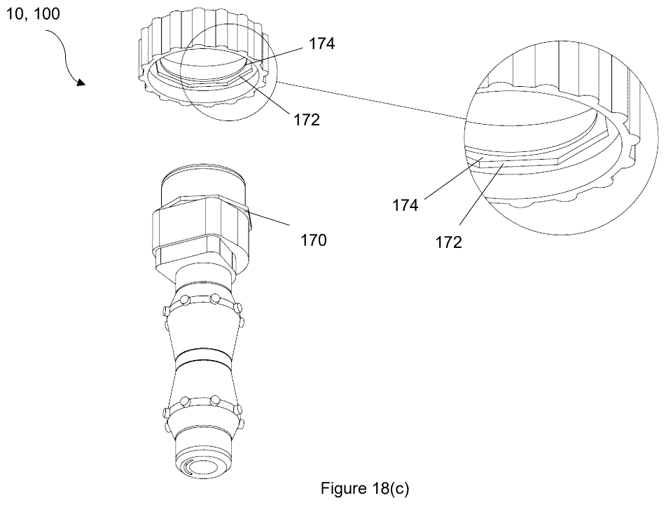

An alternative pump drive dial 160 for cannulas 10, 100 is illustrated in FIGS. 18(a)-(c). In order to minimize the size or width of the cannulas, which is advantageous when multiple cannulas are deployed close to each other, drive dial 160 has a removable component. Drive dial 160 comprises a larger removable dial 162 and an installed core 164 on the cannula body. For simplicity, the cannulas are not described in detail except for the alternative pump drive dial 160 and related components.

Drive dial 160 is usable to rotate or operate the impeller pump or the peristaltic pump or other pumps, generally referred to as pump 166 in FIGS. 18(a)-(c). Installed core 164 comprises a body portion 168 and drive hub 170. Drive hub 170 is preferably connected to drive hub 38 described above, such that both drive hubs rotate at the same time by removable dial 162. More preferably, drive hub 170 replaces drive hub 38. Removable dial 162 has driving hole 172, which is sized and dimensioned to receive drive hub 170. Both driving hole 172 and drive hub 170 have substantially the same shape and dimensions so that rotating removable dial 162 would rotate drive hub 170 to drive pump 166. Removable dial 162 also has retaining edge 174, which interferes with the body of pump 166 to prevent the removable dial from slipping beyond drive dial 170 in a distal direction.

In this alternative embodiment, cannula 10 or 100 without removable dial 162 is inserted into a surgical site. To inflate the outer membrane or dial balloon, removable dial 162 is inserted over the cannula until driving hole 172 mates with drive hub 170. A rotation, e.g., in the direction from a pump inlet to a pump outlet, to inflate the outer membrane or distal balloon. Thereafter, removable dial 162 is removed and is absent during the surgical procedure. To remove the cannula after the procedure removable dial 162 is reinserted over the cannula in the same manner and a counter-rotation would deflate the outer membrane or distal balloon.

In yet another embodiment, the impeller pump or the peristaltic pump can be redesigned with minimal alternation and within the skills of those of ordinary skill in the art to be modular. In other words, a working pump can be moved from one cannula 10, 100 to another to obviate the need to have multiple working pumps in one surgical procedure. In one example, referring to FIGS. 2 and 4(a)-(b), members 30, 32 and 34, which as a modular unit make up the impeller pump can be replaced in a second or third cannula, etc., with a false pump unit with a false impeller pump chamber 30′ and a false cap 34′ with the internal working mechanisms of the pump omitted. After a first cannula 10 with a working impeller pump is deployed, the impeller pump mechanism 30, 32, 34 is removed and replaces a false pump 30′, 34′ on the second or third cannula 10′ without a working impeller pump. This replacement is repeated until all cannulas 10, 10′ for the surgical procedure are inflated. To deflate and remove these cannulas, the same replacement is repeated in reverse.

The same modular concept can be applied to cannula 100 with the peristaltic pump. In this example, peristaltic pump 102 comprising peristaltic chamber 104, tubing assembly 106 and roller assembly 108, as best illustrated in FIGS. 11 and 14(a)-(c), is omitted and is replaced by a false peristaltic chamber 104′ and a false roller assembly 108′, preferably without the rollers 114. After completing deployment of a cannula 10 with a working peristaltic pump 102, the working pump 102 is removed and replaces the false pump in the other cannula 100′ until all cannula 100, 100′ are fully deployed. The withdrawal of cannulas in similar to that described in the preceding paragraph.

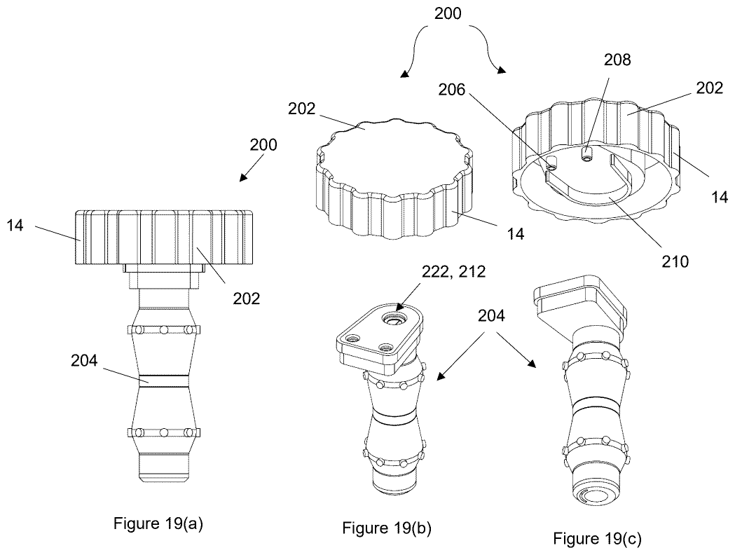

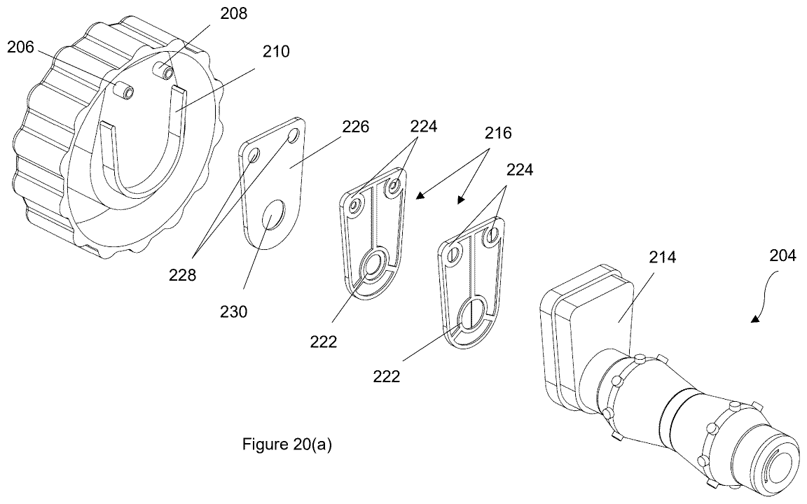

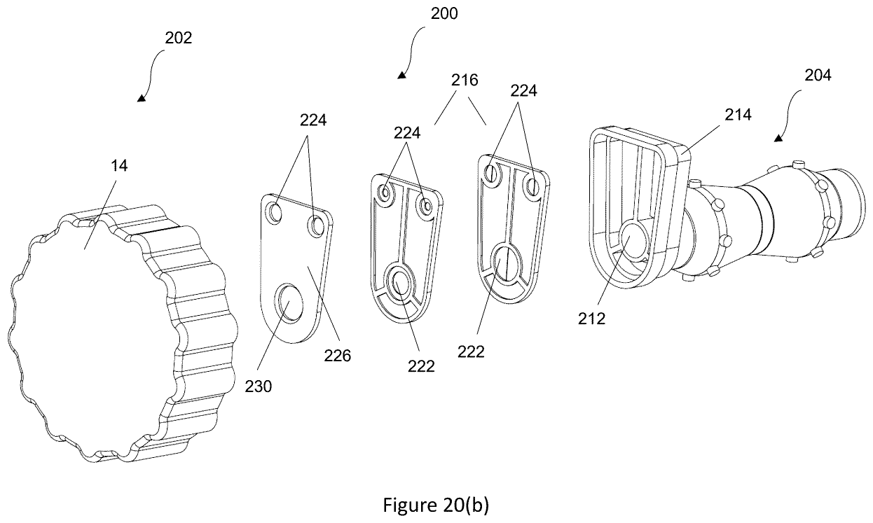

A more preferred embodiment of a modular cannula 200 is illustrated in FIGS. 19 and 20 and subparts. Cannula 200 comprises first modular part or pump assembly 202, which can include either the impeller pump or the peristaltic pump, discussed herein, and the modular cannula assembly 204, as illustrated in FIGS. 19(a)-(c). Pump assembly 202 has drive dial 14 which operates the pump enclosed in the first modular part. On the bottom surface of pump assembly 202 are pump inlet 206 and pump outlet 208, as well as optional alignment 210. Pump inlet 206 is fluidly connected to the fluid in the body cavity through a port such as inlet duct 62 and body inlet channel 148, and pump outlet 206 is fluidly connected to inflation hole(s) 64 and outer membrane 18.

Cannula assembly 204 comprises cannula body 16, which defines lumen 212 for medical instrument(s) to pass through, and outer membrane 18. Cannula assembly 204 also comprises pump receiving receptacle 214 sized and dimensioned to receive at least partially pump assembly 202, namely at least pump inlet/outlet 206, 208. Within pump receiving receptacle 214 are diaphragm assembly 216. Preferably, two diaphragm assemblies 216 are utilized, as shown. Each diaphragm assembly 216 comprises a lumen diaphragm 222 similar to diaphragms 22 of cannula 10, 100, to cover lumen 212. Each diaphragm 216 further comprises a pair of pump diaphragms 224 adapted to receive and seal pump inlet 206 and pump outlet 208. Diaphragm 222, 224 preferably comprises one diaphragm with a slit and one diaphragm with openings sized to receive pump inlet/outlet 206, 208 and seal said pump inlet/outlet and one opening sized to allow medical instrument(s) to pass through and seal the outside of the medical instrument. Pump receiving receptacle 214 further comprises a seal retainer 226, which has openings 228 to allow pump inlet/outlet 206, 208 to pass through and opening 230 to let medical instruments to pass through lumen 212.

Diaphragm assembly 216, of which two are preferred as shown, is received in pump receiving receptacle 214. Seal retainer 226 is then placed on top to complete the cannula assembly 204, as illustrated in FIG. 19(b).

To deploy cannula 200, after the incision into a body cavity is made, cannula assembly 204 is inserted into the cut tissue. Diaphragm assembly(ies) 216 with diaphragms 222 and 224 seals the fluid from the body cavity from exiting the proximal end of cannula assembly 204. Pump assembly 202 is then placed on top of cannula assembly 204 with inlet 206 and outlet 208 sealingly received in pump receiving receptacle 214 to establish a fluid communication or a fluid connection between the body cavity fluid, such as insufflated fluid, and outer membrane 18. Alignment 210 can be used to guide the connection of pump assembly 202 to cannula assembly 204. As illustrated in FIGS. 19(c) and 20(a), alignment 210's curved end fits around the outside of the corresponding curved end of pump receiving receptacle 214. Dial 14 is then rotated, as described above, to inflate outer membrane 18.

After the inflation of the outer membrane of one cannula 200, pump assembly 202 is preferably removed to provide more space for the surgeons during the procedure and is preferably used to inflate the outer membrane of other cannula assembly(ies) 204. After the surgical procedure, pump assembly 202 is re-installed to deflate the outer membrane to withdraw cannula assembly 204. It is noted that one pump assembly 202 can be utilized with multiple cannula assemblies 204, whether in a single surgical procedure or over several surgical procedures spanning a longer time period, as long as the pump assembly 202 is sanitized before each procedure.

In another alternative, inlet duct 62 and body inlet channel 148 of cannula 10 and 100, respectively may end on the outside of cannula body 16, e.g., not opening to lumen 60, so long as their terminal or port is located where outer membrane 18 does not cover.

Optionally, a locking mechanism may be used to prevent drive dial 14 from rotating after outer membrane 18 is fully inflated to prevent unintentional deflation. Exemplary locking mechanisms include but are not limited to the latching mechanisms shown in FIGS. 15 and 16 of the Mikol patents and patent applications. The present inventors believe that for the impeller pump the frictional force between terminals 46 of arms 44 and the inside surface of pump chamber 30 is sufficient to resist counter-rotation that might deflate outer membrane 18 or distal balloon. Also, for the peristaltic pump the frictional force between rollers 114 and tubes 122 is sufficient to resist counter-rotation that might deflate outer membrane 18 or distal balloon.

While it is apparent that the illustrative embodiments of the invention disclosed herein fulfill the objectives stated above, it is appreciated that numerous modifications and other embodiments may be devised by those skilled in the art. Therefore, it will be understood that the appended claims are intended to cover all such modifications and embodiments, which would come within the spirit and scope of the present invention.

What is claimed is:

1. A cannula comprising:
a cannula body defining a lumen and having a longitudinal axis,
an outer membrane covering a portion of an outside surface of the cannula body,
a pump fluidly connected to a port on the cannula body and to the outer membrane to transport a fluid through the port to inflate the outer membrane, and
a dial rotatable to actuate the pump.

2. The cannula of claim 1, wherein a rotation of the dial moves the fluid to inflate the outer membrane, and a counter-rotation of the dial reverses the flow of the fluid.

3. The cannula of claim 1, wherein the pump is an impeller pump comprising a pump chamber having a cam surface including an outlet and an inlet, and an impeller comprising a plurality of flexible radial arms that contact the pump chamber, wherein a rotation of the dial rotates the impeller causing the fluid, such as insufflated fluid, to enter the pump chamber through the inlet and exit through the outlet, wherein the inlet is fluidly connected to said port.

4. The cannula of claim 3, wherein each flexible radial arm comprises an enlarged terminal in contact with the pump chamber.

5. The cannula of claim 3, wherein the dial is keyed to the impeller to rotate the impeller as the dial is rotated.

6. The cannula of claim 1, wherein the pump is a peristaltic pump comprising a roller member, sized and dimensioned to press a flexible tube containing the fluid, such as insufflated fluid, wherein as the roller rotates, it presses against the flexible tube to move the fluid through the flexible tube and through the peristaltic pump.

7. The cannula of claim 1, wherein the pump is a peristaltic pump comprising a peristaltic chamber, a tubing assembly and a roller assembly, wherein the tubing assembly is positioned inside the peristaltic chamber and the roller assembly is located inside the tubing assembly.

8. The cannula of claim 7, wherein the roller assembly comprises at least one roller rotatably mounted on a roller hub.

9. The cannula of claim 8, wherein the tubing assembly comprises at least one tube fluidly connected to the lumen and the outer membrane.

10. The cannula of claim 9, wherein when rotated the at least one roller presses the at least one tube to transport the fluid.

11. The cannula of claim 7, wherein the peristaltic chamber comprises an inlet and an outlet, wherein the inlet is fluidly connected to the lumen or is located at a distal end of the cannula body and the outlet is fluidly connected to the outer membrane, wherein the inlet is fluidly connected to said port.

12. The cannula of claim 7, wherein the dial is keyed to the roller assembly to rotate the roller assembly as the dial is rotated.

13. The cannula of claim 1 further comprising a sealing cap with a diaphragm to cover the lumen.

14. The cannula of claim 13, wherein the diaphragm comprises at least two disks each having a slit cut thereon, wherein the at least two disks are arranged so that the slits are at an angle to each other and wherein the slits are arranged to be aligned with the lumen.

15. The cannula of claim 13, wherein the diaphragm comprises at least two disks, wherein one disk has a slit cut thereon and the other disk has an opening that is sized to allow a medical instrument to pass through while maintaining a seal with an outer surface of the medical instrument.

16. The cannula of claim 1, wherein the outer membrane cover a significant portion of the cannula body, preferably more than a third, more preferably more than half of the cannula body.

17. The cannula of claim 1, wherein the outer membrane has a dog bone or hour-glass shape.

18. The cannula of claim 1, wherein the outer membrane has an outer micro-textured surface, or the outer membrane has at least one macro texture such as a knob.

19. The cannula of claim 1, wherein the outer membrane is a distal balloon.

20. The cannula of claim 1, wherein the rotatable dial comprises an installed core and a removable dial, wherein the removable dial rotates a corresponding drive hub to operate the pump.

21. The cannula of claim 1, wherein said port opens to the lumen or to a distal end of the cannula body.

22. The cannula of claim 1, wherein the pump is modular, wherein the pump and the dial form a first modular part and a second modular part comprises the cannula body, the outer membrane and a pump receiving receptacle.

23. The cannula of claim 1, wherein its axis of rotation is parallel to or coincide with the longitudinal axis of the cannula body.
